(19) United States
(12) Patent Application Publication
(10) Pub. No.: US 2023/0320695 A1
(43) Pub. Date: Oct. 12, 2023

(54) CATHETER-TYPE ULTRASOUND ENDOSCOPE AND EXAMINATION SYSTEM INCLUDING SAME

(71) Applicants: INDUSTRY FOUNDATION OF CHONNAM NATIONAL UNIVERSITY, Gwangju (KR); KOREA INSTITUTE OF MEDICAL MICROROBOTICS, Gwangju (KR); INDUSTRY-ACADEMIC COOPERATION FOUNDATION, YONSEI UNIVERSITY, Seoul (KR)

(72) Inventors: Chang Sei KIM, Gwangju (KR); Jong Oh PARK, Gyeonggi-do (KR); Byung Jeon KANG, Gwangju (KR); Seung Min BANG, Seoul (KR); Jae Hee CHO, Seoul (KR); Moon Jae CHUNG, Seoul (KR); Ja Young KIM, Daejeon (KR); Sung Hwan CHUNG, Gwangju (KR)

(21) Appl. No.: 18/022,902

(22) PCT Filed: Aug. 23, 2021
(86) PCT No.: PCT/KR2021/011174

§ 371 Date: Feb. 23, 2023

(30) Foreign Application Priority Data
Aug. 24, 2020 (KR) 10-2020-0105872

(51) Int. Cl. A61B 8/00; A61B 10/04

(52) U.S. Cl. CPC A61B 8/445; A61B 8/4461; A61B 10/04; A61B 2010/045

(57) ABSTRACT

A catheter-type ultrasound endoscope according to an embodiment of the present invention includes an ultrasound module for acquiring an ultrasound image, and a biopsy needle, wherein the ultrasound module includes a pMUT module and an ASIC signal processing circuit, and the ASIC signal processing circuit uses CMOS-MEMS technology. This steerable catheter-type ultrasound endoscope capable of performing tissue biopsies according to the present invention obtains ultrasound images from representative digestive organs, such as the bile duct and the pancreatic duct, through the miniaturization of the ultrasound module, and performs a biopsy while the ultrasound images are observed in real time, and thus can improve the accuracy of diagnosis.

### TECHNICAL FIELD

The present disclosure has been made with the support of the ministry of Science and ICT, the ministry of Trade, Industry and Energy, the ministry of Health and Welfare, and the Ministry of Food and Drug Safety of the Republic of Korea under Project ID. No. 9991006822, and sub-project No. 202012E11-01, which was conducted by the Korea Institute of Medical Microrobotics in the research program named “Development of Catheter-Type Ultrasound Endoscope Micro-MediBot for Diagnosis of Gastrointestinal Diseases” as a branch of the research project titled “Korea Medical Device Development Project (R&D)” under the research management of the Korea Medical Device Development Fund, from Sep. 01, 2020 to Feb. 28, 2021.

This application claims priority to and the benefit of Korean Application Number 10-2020-0105872, filed in the Korean Intellectual Property Office on Aug. 24, 2020, the entire content of which is incorporated herein by reference.

The present disclosure relates to a catheter-type ultrasound endoscope that is steerable and enables a tissue biopsy. More specifically, the present disclosure relates to a catheter-type ultrasound endoscope including a channel that can accommodate a tissue biopsy needle or other operational devices through miniaturization of an ultrasound module.

### BACKGROUND ART

In general, a catheter is a tubular device designed to be inserted into a body cavity or an organ such as the stomach, intestines, bladder, and so on to diagnose a condition or to inject nutrients or medicines, and is mainly used for medical purposes. There are conventional sonographies that are capable of visualizing the inside of the digestive tract after being inserted through the oral cavity, such as intraductal ultrasound (hereinafter referred to as “IDUS”) and endoscopic ultrasound (hereinafter referred to as “EUS”).

However, conventional IDUS and EUS are difficult to miniaturize because of the employment of ultrasonic transducers manufactured by mechanically processing piezoelectric ceramics, and have to perform signal processing outside the catheter.

Previously developed IDUS failed to secure a channel into which a biopsy needle could be inserted and acquired only ultrasound images perpendicular to the direction of catheter travel, making additional procedures such as biopsy impossible. In addition, EUS had a problem in that it was unable to enter into a lumen because it could not be miniaturized.

In order to solve such problems, the use of a MEMS device and an ASIC signal processing circuit is required to implement an ultrasound array capable of signal processing inside the catheter.

Recently, an intracardiac ultrasound probe using MEMS technology has been successfully manufactured, but the high rigidity of the catheter due to many signal processing lines therein makes it difficult to sufficiently secure steering for the probe and does not guarantee a space into which other channels can enter.

### DETAILED DESCRIPTION OF THE INVENTION

### Technical Problem

The present disclosure aims to provide a catheter-type ultrasound endoscope that is steerable and capable of performing a tissue biopsy. Specifically proposed herein is a catheter-type ultrasound endoscope that is sufficiently steerable and miniaturized and allows for additional therapeutic intervention while acquiring ultrasound images.

### Technical Solution

An aspect of the present disclosure is drawn to a catheter-type ultrasound endoscope which includes: an ultrasound module for acquiring an ultrasound image; a biopsy needle; and an electromagnetic rotation part for rotating the catheter-type ultrasound endoscope using an internal micromotor, wherein the ultrasound module is configured by combining a pMUT chip and an ASIC chip.

In accordance with an embodiment, the catheter-type ultrasound endoscope further includes a guide wire.

In accordance with an embodiment, the catheter-type ultrasound endoscope is 3.3 mm or less in diameter.

In accordance with an embodiment, the catheter-type ultrasound endoscope is characterized in that the ultrasound module acquires an ultrasound image by generating ultrasound in a lateral direction of the catheter travel.

In accordance with an embodiment, the catheter-type ultrasound endoscope is characterized in that the endoscope unit generates ultrasound so that the biopsy unit is displayed on the ultrasound image.

Another aspect of the present disclosure pertains to an ultrasound endoscopy system including: a catheter-type ultrasound endoscope; and an inspection part for controlling the catheter-type ultrasound endoscope, wherein the catheter-type ultrasound endoscope includes: an ultrasound module for acquiring an ultrasound image; and a biopsy needle, the ultrasound module being configured with a pMUT module and an ASIC signal processing circuit, and the inspection part (210) includes: an ultrasound endoscope controller; and an ultrasound endoscopic image display part.

### Advantageous Effects

According to the present disclosure, as described above, the advantages described below can be obtained. However, the advantages obtainable through the present invention are not limited thereto.

Having a miniaturized ultrasound module, the catheter-type ultrasound endoscope that is steerable and capable of performing a tissue biopsy according to the present invention can obtain ultrasound images from typical digestive organs, such as the bile duct and pancreatic duct, and perform a biopsy while checking the image in real-time, thereby ensuring the reliability of diagnosis.

In addition, the catheter-type ultrasound endoscope has the advantage of being able to check the position of the tissue biopsy needle by generating ultrasound in the lateral (azimuth) direction.

### BRIEF DESCRIPTION OF THE DRAWINGS

FIG. 1 is an exemplary view of a catheter-type ultrasound endoscope including a miniaturized ultrasound module according to an embodiment of the present disclosure.

FIG. 2 is a view showing a structure of the ultrasound module according to an embodiment of the present disclosure.

FIG. 3 is a different view of a structure of the ultrasound module according to an embodiment of the present disclosure.

FIG. 4 shows cross-sectional views of the catheter-type ultrasound endoscope including an internal micromotor according to an embodiment of the present disclosure.

FIG. 5 is an exemplary view illustrating control of directions of the ultrasound endoscope through external magnetic fields according to an embodiment of the present disclosure.

FIG. 6 is a view of an ultrasound endoscopy system including a catheter-type ultrasound endoscope and an inspection part for controlling the catheter-type ultrasound endoscope with an electromagnetic coil according to an embodiment of the present disclosure.

FIG. 7 is a view showing performance comparison between the ultrasound endoscope of the present disclosure and conventional endoscopic ultrasound (EUS)/intraductal ultrasound (IDUS).

### BEST MODE FOR CARRYING OUT THE INVENTION

Disclosed herein is a catheter-type ultrasound endoscope which includes: an ultrasound module for acquiring an ultrasound image; a biopsy needle; and an electromagnetic rotation part for rotating the catheter-type ultrasound endoscope using an internal micromotor, wherein the ultrasound module is configured by combining a pMUT chip and an ASIC chip.

### MODE FOR CARRYING OUT THE INVENTION

Hereinafter, exemplary embodiments according to the present invention will be described in detail below with reference to the accompanying drawings. The detailed description to be given below with reference to the accompanying drawings is for describing exemplary embodiments of the present disclosure and is not intended to represent the only embodiment in which the present disclosure may be practiced.

The embodiments are provided to make the disclosure of the present invention complete and completely inform one of ordinary skill in the art to which the present invention pertains of the scope of the invention, and the present invention is defined only by the scope of the claims below.

In some cases, to avoid blurring the concept of the present invention, known structures and devices may be omitted from the drawings, or a structure and a device may be illustrated in the form of a block diagram focused on key functions of the structure and the device. Also, throughout the present specification, like elements are denoted by like reference numerals.

Throughout the specification, when a certain part is described as “comprising” or “including” a certain element, this signifies that the certain part may also include another element rather than excluding the other element unless particularly described otherwise.

In the present specification, the term “part” refers to a unit of processing one or more functions or operations, and a “part” may be implemented with hardware, software, or a combination of hardware and software. Further, terms such “a”, “an”, “one”, and other similar related words may be used as including both singular and plural meanings unless indicated otherwise herein in the context of the present invention or clearly contradicted by the context.

Further, specific terms used in the embodiments of the present invention are provided to assist understanding of the present invention, and unless defined otherwise, all terms used herein including technical or scientific terms have the same meanings as those generally understood by one of ordinary skill in the art to which the present invention pertains. The use of such specific terms may be changed in other forms within the scope not departing from the technical spirit of the present invention.

Hereinafter, exemplary embodiments according to the present invention will be described in detail below with reference to the accompanying drawings. The detailed description to be given below with reference to the accompanying drawings is for describing exemplary embodiments of the present invention and is not intended to represent the only embodiment in which the present invention may be practiced.

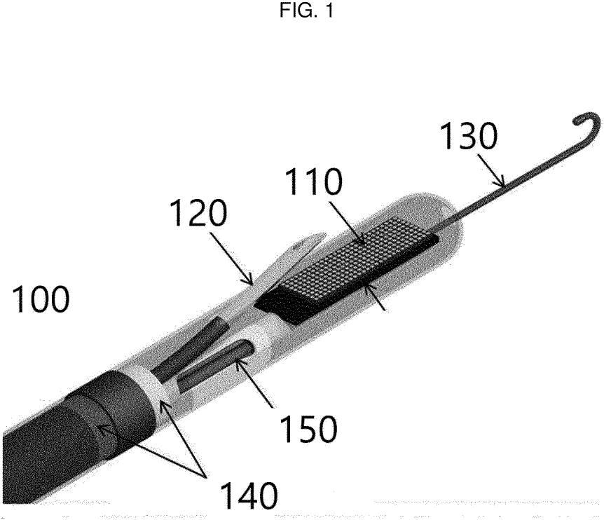

FIG. 1 is an exemplary view of a catheter-type ultrasound endoscope including a miniaturized ultrasound module according to an embodiment of the present disclosure.

The catheter-type ultrasound endoscope of the present disclosure may include an ultrasound module (110), a biopsy needle (120), a guide wire (130), an electromagnetic rotation part (140), and an electric wire (150).

The ultrasound module (110) is adapted for acquiring ultrasonic images of organs in the body. The ultrasound module (110) includes a circuit integrated using CMOS-MEMS technology. Accordingly, the ultrasound module (110) is smaller in size than conventional ultrasound endoscopes and thus can enter a lumen and directly travel the digestive tract.

The biopsy needle (120) is adapted for sampling tissues or cells. The biopsy needle (120) is used to implement examinations, such as fine needle aspiration biopsy, etc., depending on histo- or cytomorphology.

The guide wire (130) is an extremely thin steel wire to be inserted into the body to guide the catheter-type ultrasound endoscope to blood vessels. With the steel wire at the center, the guide wire (130) is structured to have an extremely thin spring steel wire spirally wrapping around the core and may be in the form where a very elastic and flexible semicircular steel wire is welded to the end of the guide wire for safety. Processed with Teflon, the guide wire (130) is smooth and unlikely to be broken or bent: the guide wire, if tough or broken and bent in the blood vessels during an examination, may damage the blood vessels. The guide wire (130) may be configured to have various dimensions such as 70-220 cm in length and 0.635(0.025″)-1.100 mm (0.045″) in diameter. The guide wire (130) may be terminated with a semicircular form, such as “J” form, or a “linear” (straight) form.

The electromagnetic rotation part (140) is adapted for rotating the catheter-type ultrasound endoscope (100). The catheter-type ultrasound endoscope (100) of the present disclosure is configured to check the position of the tissue biopsy needle by generating ultrasonic wavelengths in a lateral direction, thereby requiring a rotation function to acquire lateral endoscope views from various angles. Accordingly, the electromagnetic rotation part (140) includes an internal micromotor and can be rotated 360 degrees in all directions, such as clockwise and counterclockwise. The internal micromotor of the electromagnetic rotation part (140) may be configured to include a micro-coil and rotate depending on the influence of an external electromagnetic field.

The electric wire (150) may be used to control the operation of the above-described components of the catheter-type ultrasound endoscope (100), supply power, and transmit endoscopic image data acquired through the ultrasound module (110).

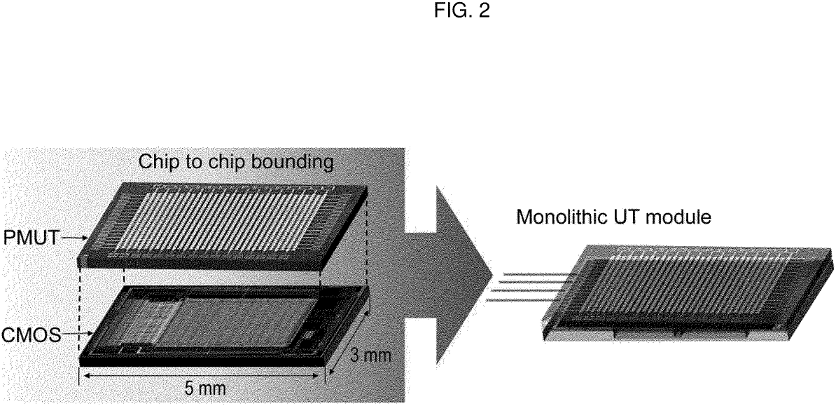

FIG. 2 is a view showing a structure of the ultrasound module according to an embodiment of the present disclosure.

The ultrasound module (110) may be configured by combining a pMUT chip (111) and an ASIC (112) chip. The pMUT chip (111) and the CMOS chip (112) are circuits for acquiring ultrasound endoscopic images.

The pMUT chip (111) is a piezoelectric micromachined ultrasonic transducer that is a MEMS-based piezoelectric ultrasonic transducer. Unlike bulk piezoelectric transducers that use the thickness-mode operation of a piezoelectric ceramic plate, the pMUT is based on the bending operation of a thin-film piezoelectric layer. Compared to bulk piezoelectric ultrasonic transducers, pMUTs can offer advantages such as low acoustic impedance, low voltage usage, and integration with signal processing circuitry.

The ASIC (112) chip is a customized system semiconductor manufactured according to the order for a specific purpose, and is widely used for the production of high-tech products due to its high reliability and high-speed processing.

The ASIC (112) chip, which is a device manufactured by complementary metal oxide semiconductor (CMOS) technology, has numerous circuits integrated on a silicon substrate and thus can process many signals simultaneously in a small chip. The ASIC (112) chip can be miniaturized compared to a circuit constructed on a printed circuit board, and is used in most electronic devices today due to its low delay and low power consumption.

The ASIC 112 chip of the present disclosure may be manufactured according to the purpose of obtaining an ultrasound image.

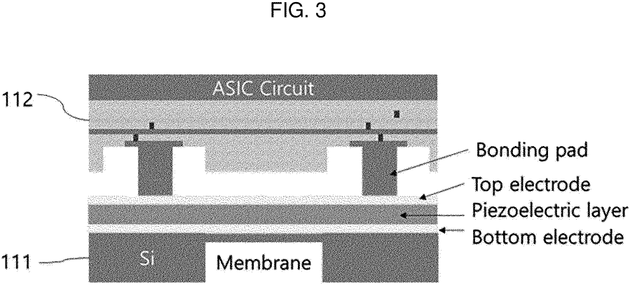

FIG. 3 is a different view of a structure of the ultrasound module according to an embodiment of the present disclosure.

In the ultrasound module (110), the pMUT chip (111) and the ASIC chip (112) may be combined to each other via a bonding pad. The bonding pad exhibits an electrode pad, which may be composed of eutectic bonding or solder bumps, on a semiconductor chip. In addition, a piezoelectric layer may be located between the pMUT chip (111) and the ASIC chip (112), and a top electrode, a bottom electrode, and an elastic layer may be disposed on and beneath the piezoelectric layer.

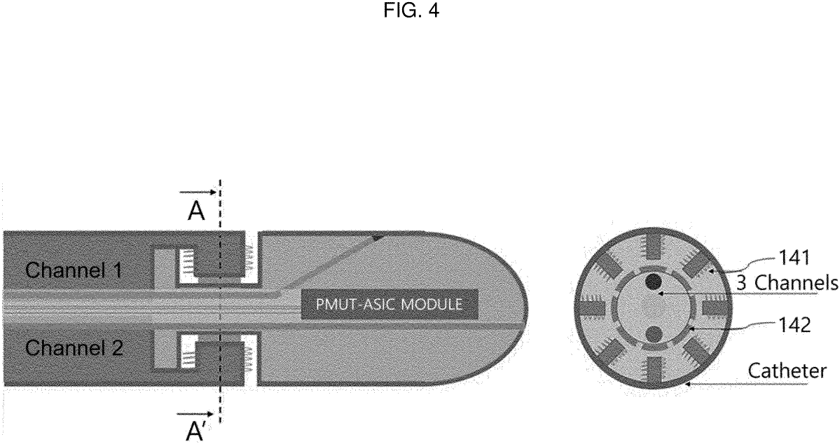

FIG. 4 shows cross-sectional views of the catheter-type ultrasound endoscope including an internal micromotor according to an embodiment of the present disclosure.

As can be seen in FIG. 4, the catheter-type ultrasound endoscope (100) may include three channels in the center. The three channels may be configured for various purposes such as the ultrasound module (110), the biopsy needle (120), and the guide wire (130). Permanent magnets (142) may be positioned outside the three channels, and micro coils (141) may be correspondingly arranged outside the permanent magnets (142). The micro coils (141) and the permanent magnets (142) account for the electromagnetic rotation part (140) for rotating the catheter-type ultrasound endoscope (100).

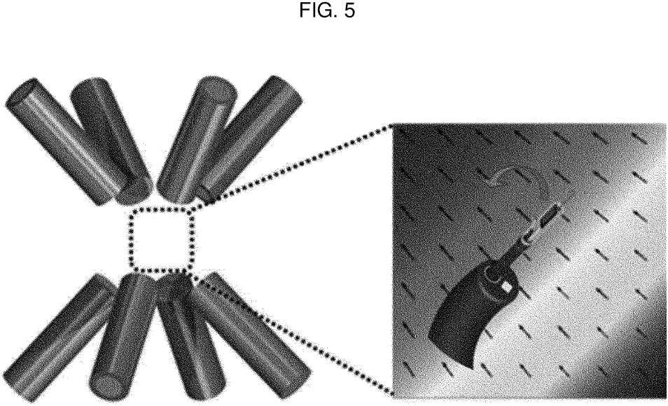

FIG. 5 is an exemplary view illustrating control of directions of the ultrasound endoscope through external magnetic fields according to an embodiment of the present disclosure. The catheter-type ultrasound endoscope (100) can be integrated into a conventional endoscope channel and used as an accessory to an existing hospital endoscope system including a manual operation method.

The catheter-type ultrasound endoscope (100) including the electromagnetic rotation part (140) may be rotated by an external magnetic field. For example, as shown in FIG. 5, when an external magnetic field is formed in an upward diagonal direction to the left, the catheter-type ultrasound endoscope (100) may be configured to rotate counterclockwise. In such a way, the operator can control the rotation of the catheter-type ultrasound endoscope (100) inside the subject’s body to obtain a desired image by adjusting the direction and strength of the external magnetic field.

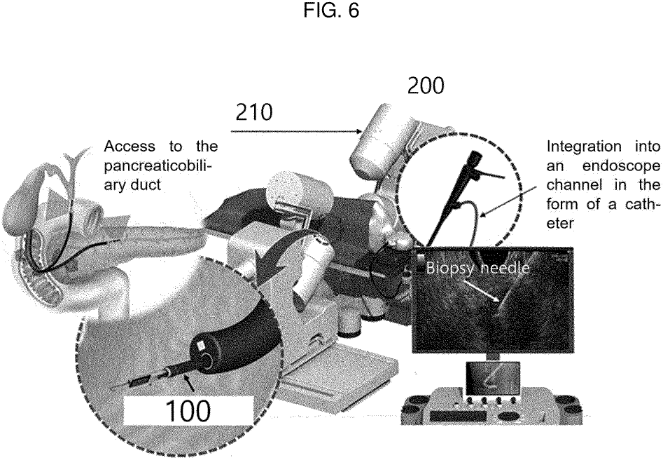

FIG. 6 is a view of an ultrasound endoscopy system including a catheter-type ultrasound endoscope and an inspection part for controlling the catheter-type ultrasound endoscope with an electromagnetic coil according to an embodiment of the present disclosure.

The ultrasound endoscopy system (200) may be configured to include the above-described catheter-type ultrasound endoscope (100) and an inspection part (210) for controlling same.

The inspection part (210) may include an external magnetic field generation part, an ultrasound endoscope controller, and an ultrasound endoscopic image display part.

By adjusting the direction and intensity of the external magnetic field, the catheter-type ultrasound endoscope (100) can be controlled. The ultrasound endoscopy system (200) can control the movement of the catheter-type ultrasound endoscope (100) within the human body and display the ultrasound endoscopic images acquired from the catheter-type ultrasound endoscope (100). In addition, the catheter-type ultrasound endoscope (100) may control overall matters through the ultrasound endoscope, such as performing a biopsy with reference to the acquired ultrasound endoscopic images.

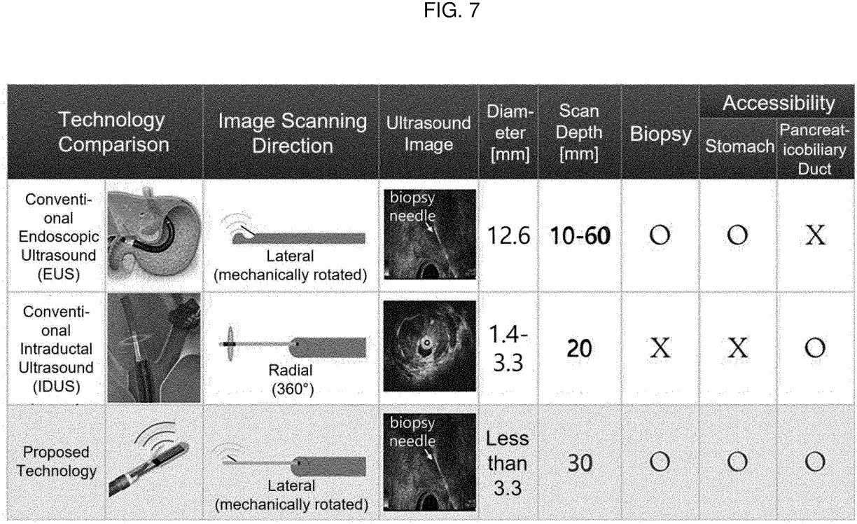

FIG. 7 is a view showing performance comparison between the ultrasound endoscope of the present disclosure and conventional endoscopic ultrasound (EUS)/intraductal ultrasound (IDUS).

Conventional endoscopic ultrasound (EUS) has a lateral image scanning direction through ultrasounds, can be mechanically rotated, allows for biopsy performance, and enables access to the stomach. However, it is 12.6 mm in diameter and thus cannot access the inside of the pancreaticobiliary duct.

Conventional intraductal ultrasound (IDUS) has a radial direction of image scanning through ultrasound and a diameter of 1.4-3.3 mm, allowing access to the pancreaticobiliary duct. However, since radial ultrasound images are acquired, it is difficult to determine the location of the biopsy needle, and thus has a limitation in practical use.

In contrast, the catheter-type ultrasound endoscope (100) proposed above has a lateral image scan direction through ultrasound and can be rotated mechanically. Furthermore, the catheter-type ultrasound endoscope (100) is composed of a catheter with a diameter of 3.3 mm including the tissue biopsy needle (120) through miniaturization of the ultrasound module, enabling tissue biopsy and access to the stomach and pancreaticobiliary duct.

Therefore, the catheter-type ultrasound endoscope (100) proposed above is capable of acquiring ultrasound images and tissue biopsies inside the pancreaticobiliary duct, which is impossible with conventional endoscopic ultrasound (EUS), and performing tissue biopsies that cannot be done with intraductal ultrasound (IDUS). The catheter-type ultrasound endoscope can thus be used for accurate clinical diagnosis in the digestive system.

One of ordinary skill in the art related to the embodiments of the present invention should understand that the present disclosure may be implemented in modified forms within the scope not departing from the essential feature of the above description. Therefore, the methods disclosed herein should be taken into consideration in terms of an illustrative aspect instead of a limiting aspect. The scope of the present invention is defined by the claims below instead of the detailed description of the invention, and all differences within the scope equivalent to the claims below should be interpreted as belonging to the scope of the present invention.

### INDUSTRIAL APPLICABILITY

The present disclosure pertains to a catheter-type ultrasound endoscope that is steerable and enables a tissue biopsy. More specifically, the present disclosure relates to a catheter-type ultrasound endoscope including a channel that can accommodate a tissue biopsy needle or other operational devices through miniaturization of an ultrasound module.

What is claimed is:

1. A catheter-type ultrasound endoscope, comprising:
an ultrasound module for acquiring an ultrasound image;
a biopsy needle; and
an electronic rotation part for rotating the catheter-type ultrasound endoscope using an internal micromotor,
wherein the ultrasound module is configured by combining a pMUT chip and an ASIC chip.

2. The catheter-type ultrasound endoscope of claim 1, wherein the catheter-type ultrasound endoscope further comprises a guide wire.

3. The catheter-type ultrasound endoscope of claim 1, wherein the catheter-type ultrasound endoscope is 3.3 mm or less in diameter.

4. The catheter-type ultrasound endoscope of claim 1, wherein the ultrasound module acquires an ultrasound image by generating ultrasound in a lateral direction of the catheter travel.

5. The catheter-type ultrasound endoscope of claim 4, wherein the ultrasound module generates ultrasound so that the biopsy needle is displayed on the ultrasound image.

6. An ultrasound endoscopy system, comprising:
a catheter-type ultrasound endoscope; and
an inspection part for controlling the catheter-type ultrasound endoscope,
wherein the catheter-type ultrasound endoscope comprises: an ultrasound module for acquiring an ultrasound image; and a biopsy needle, the ultrasound module being configured with a pMUT module and an ASIC signal processing circuit, and the inspection part comprises: an ultrasound endoscope controller; and an ultrasound endoscopic image display part.
